(19) United States
(12) Patent Application Publication
(10) Pub. No.: US 2016/0113861 A1
(43) Pub. Date: Apr. 28, 2016

(54) HAIRCARE COMPOSITION

(71) Applicant: Keisha Wilson, MISSOURI CITY, TX (US)

(72) Inventor: Keisha Wilson, MISSOURI CITY, TX (US)

(21) Appl. No.: 14/521,352

(22) Filed: Oct. 22, 2014

(51) Int. Cl. A61K 8/97; A61K 8/23; A61K 8/34; A61K 8/67; A61K 8/92; A61Q 7/00

(52) U.S. Cl. CPC A61K 8/97; A61K 8/927; A61Q 7/00; A61K 8/925; A61K 8/34; A61K 8/678; A61K 8/23; A61K 8/673

(57) ABSTRACT

Some embodiments of the present disclosure include a haircare composition for preventing hair loss and promoting hair regrowth. The haircare composition may include a base composition including lanolin, shea butter, coconut oil, and beeswax, a moisturizer including olive oil, Vitamin E oil, apricot oil, castor oil, and grape-seed oil, a stimulant including menthol, gooseberry extract, sulfur, Vitamin B complex, and black seed oil, and a strengthener including sage essential oil, rosemary essential oil, and peppermint essential oil. The haircare composition may regrow and strengthen hair, resolve baldness, and repair damaged hair follicles.

### BACKGROUND

The embodiments herein relate generally to haircare, and more particularly, to a hair regrowth and restoration composition and system.

Hair loss is common with aging and seemingly inevitable to many. Hair loss is also associated with certain diseases or medical treatments. For example, many cancer patients have to undergo chemotherapy, which is known to result in hair loss. Similarly, alopecia, hair fungi, and other skin/scalp trauma can cause hair thinning or loss.

Conventional hair products promise results, but seldom work for women and most do not work for men at all. Specifically, hair loss is contingent on many contributing factors and most products simply fail to prevent hair loss or promote regrowth of hair, because the products fail to cater to all hair types and gender.

Therefore, what is needed is haircare composition for preventing hair loss and promoting regrowth of the hair that works with a variety of hair types, while also being safe for use by people, including those suffering from alopecia, hair fungi, and cancer.

### SUMMARY

Some embodiments of the present disclosure include a haircare composition for preventing hair loss and promoting hair regrowth. The haircare composition may include a base composition including lanolin, shea butter, coconut oil, and beeswax, a moisturizer including olive oil, Vitamin E oil, apricot oil, castor oil, and grape-seed oil, a stimulant including menthol, gooseberry extract, sulfur, Vitamin B complex, and black seed oil, and a strengthener including sage essential oil, rosemary essential oil, and peppermint essential oil. The haircare composition may regrow and strengthen hair, resolve baldness, and repair damaged hair follicles.

### DETAILED DESCRIPTION OF CERTAIN EMBODIMENTS

In the following detailed description of the invention, numerous details, examples, and embodiments of the invention are described. However, it will be clear and apparent to one skilled in the art that the invention is not limited to the embodiments set forth and that the invention can be adapted for any of several applications.

The device of the present disclosure may be used to prevent hair loss and promote regrowth of hair and may comprise the following elements. This list of possible constituent elements is intended to be exemplary only, and it is not intended that this list be used to limit the haircare composition of the present application to just these elements. Persons having ordinary skill in the art relevant to the present disclosure may understand there to be equivalent elements that may be substituted within the present disclosure without changing the essential function or operation of the device.
1. Base
2. Moisturizer
3. Growth Stimulant
4. Hair Strengthener

The various elements of the haircare composition for preventing hair loss and promoting hair regrowth of the present disclosure may be related in the following exemplary fashion. It is not intended to limit the scope or nature of the relationships between the various elements and the following examples are presented as illustrative examples only.

By way of example, some embodiments of the haircare composition of the present disclosure comprise a base, at least one moisturizer, at least one hair strengthener, which may also thicken and lengthen hair, and at least one stimulant, wherein each of the components is organic and the haircare composition regrows, strengthens, and thickens hair. The haircare composition may resolve baldness and may repair damaged follicles. The combination of components in the composition may prevent thinking, breaking and hair loss and may work for various hair types and for each gender.

The base of the composition may comprise a plurality of base ingredients selected from the group consisting of lanolin, shea butter, coconut oil, beeswax, and mixtures thereof. In an exemplary embodiment, the base comprises organic lanolin, organic shea butter, organic food grade fractionated coconut oil, and organic beeswax. The organic lanolin may be dissolved, while the organic shea butter, the organic food grade fractionated coconut oil, and the organic beeswax may all be in solid form. In some embodiments, the ratio of lanolin: shea butter: coconut oil: beeswax may be about 1:1:0.5:0.5. For example, the base may comprise about ½ cup organic lanolin (dissolved), about ½ cup organic shea butter (solid), about ¼ cup organic food grade fractionated coconut oil (solid), and about ¼ cup organic beeswax (solid).

The moisturizers included in the haircare composition may comprise moisturizers selected from the group consisting of olive oil, Vitamin E oil, apricot oil, castor oil, grape-seed oil, and mixtures thereof. Some of the oils may double as softeners. In an exemplary embodiment, the haircare composition may comprise organic virgin olive oil, Vitamin E oil, apricot oil, African organic castor oil, and grape seed oil, wherein the apricot oil, African organic castor oil, and grape-seed oil may function as both moisturizers and softeners. In embodiments, the Vitamin E oil apricot oil, African organic castor oil, and grape-seed oil may be present in about the same amount by volume. For example, the haircare composition may comprise about ⅛ teaspoon (tsp) Vitamin E oil, about ⅛ tsp apricot oil, about ⅛ tsp African organic castor oil, and about ⅛ tsp grape-seed oil along with about ¼ tsp organic virgin olive oil.

The haircare composition may comprise a variety of stimulants selected from the group consisting of growth stimulants, super stimulants, and mixtures thereof. The stimulants may double as circulation and oxygen promoters as well as hair food. An exemplary growth stimulant comprises menthol, such as organic food grade menthol. An exemplary super stimulant comprises sulfur, such as organic food grade sulfur. Other exemplary stimulants comprise gooseberry extracts, such as Indian gooseberry extract, liquid Vitamin B complex, and black see oil. In an exemplary embodiment, the haircare composition may comprise a variety of stimulants comprising about ⅛ tsp organic food grade menthol, about ⅛ tsp Indian gooseberry extract, about 1/16 tsp organic food grade sulfur, about ⅛ tsp liquid Vitamin B complex, and about ⅛ tsp black seed oil. However, other embodiments of the haircare composition may not comprise any black seed oil.

The haircare composition of the present disclosure may also comprise at least one strengthener, which may strengthen, thicken, and lengthen hair. The strengthener may be selected from the group consisting of sage essential oil, rosemary essential oil, peppermint essential oil, and mixtures thereof. When multiple strengtheners are included in the haircare composition, they may be included at substantially the same amount. For example, some embodiments of the haircare composition may comprise about 1/16 tsp sage essential oil, about 1/16 tsp rosemary essential oil, and about 1/16 tsp peppermint essential oil.

The haircare composition of the present disclosure may also comprise a number of optional ingredients, which may increase the speed at which the haircare composition works. For example, the haircare composition may further comprise at least one of magnesium liquid vitamins, sarsaparilla, and organic basil.

An exemplary embodiment of the haircare composition of the present invention may comprise about ½ cup organic lanolin dissolve, about 1.2 cup organic shea butter solid, about ¼ cup organic food grade fractionated coconut oil, about ¼ cup organic beeswax solid, about ¼ tbsp. organic virgin olive oil, about ⅛ tsp organic food grade menthol, about 1/16 tsp sage essential oil, about 1/16 tsp rosemary essential oil, about 1/16 tsp peppermint essential oil, about ⅛ tsp Indian gooseberry extract, about ⅛ tsp Vitamin E oil, about 1/16 tsp organic food grade sulfur, about ⅛ tsp apricot oil, about ⅛ tsp African organic castor oil, about ⅛ tsp grape-seed oil, about ⅛ tsp liquid Vitamin B complex, and about ⅛ tsp black seed oil. While exemplary embodiments and amounts of components have been described above, the amount of each ingredient used may be scaled up or down to create smaller or larger batches of the haircare composition. Additionally, the ratio of the components may be altered to provide haircare compositions having varying strengths. For example, a combination of the listed ingredients may create: (i) a Root Repair Maximum Strength product, which may be the strongest formula for traumatized scalp conditions; (ii) a Normal Strength product, which may be a milder version than the Root Repair product and may thicken, lengthen, and strengthen hair; (iii) a Mild Stimulation Formula, which may be mildest version of the haircare composition; (iv) a Hair Growth Shampoo, which may promote hair regrowth while adding moisture and shine without greasiness; (v) a Conditioner, which may stabilize the hair shaft and support and reinforce the hair follicles; (vi) an Eyebrow Magic, which may grow eyebrow hair back thicker and stronger, and (vii) a Waver Rescue, which may help a weave last longer by reviving the texture, wherein all versions may work with all types of human hair.

To make the haircare composition of the present disclosure, the ingredients may be added to a mixing bowl, such as a large stainless steel mixing bowl, mixed well using a glass or stainless steel stirrer, and refrigerated for best results. To use the haircare composition of the present disclosure, a user may apply a small, pea-sized amount of the product to their scalp for about an hour with a plastic cap or for about 30 minutes under a dryer. The product may then be washed out, and application may be repeated about once a week or as otherwise needed.

Persons of ordinary skill in the art may appreciate that numerous design configurations may be possible to enjoy the functional benefits of the inventive systems. Thus, given the wide variety of configurations and arrangements of embodiments of the present invention the scope of the invention is reflected by the breadth of the claims below rather than narrowed by the embodiments described above.

What is claimed is:

1. A haircare composition for preventing hair loss and promoting hair regrowth, the haircare composition comprising:
a base composition comprising a base ingredient selected from the group consisting of lanolin, shea butter, coconut oil, beeswax, and mixtures thereof;
a moisturizer selected from the group consisting of olive oil, Vitamin E oil, apricot oil, castor oil, grape-seed oil, and mixtures thereof;
a stimulant selected from the group consisting of menthol, gooseberry extract, sulfur, Vitamin B complex, black seed oil, and mixtures thereof; and
a strengthener selected from the group consisting of sage essential oil, rosemary essential oil, peppermint essential oil, and mixtures thereof,
wherein the haircare composition is configured to regrow and strengthen hair, resolve baldness, and repair damaged hair follicles.

2. The haircare composition of claim 1, wherein the base comprises a mixture of lanolin, shea butter, coconut oil, and beeswax.

3. The haircare composition of claim 1, wherein the moisturizer comprises olive oil, Vitamin E oil, apricot oil, castor oil, and grape-seed oil.

4. The haircare composition of claim 1, wherein the stimulant comprises menthol, gooseberry extract, sulfur, and Vitamin B complex.

5. The haircare composition of claim 4, wherein the stimulant further comprises black seed oil.

6. The haircare composition of claim 1, wherein the strengthener comprises sage essential oil, rosemary essential oil, and peppermint essential oil.

7. The haircare composition of claim 1, further comprising at least one member selected from the group consisting of magnesium liquid vitamins, sarsaparilla, organic basil, and mixtures thereof.

8. A haircare composition for preventing hair loss and promoting hair regrowth, the composition comprising:
about ½ cup organic lanolin dissolved;
about ½ cup organic shea butter solid;
about ¼ cup organic food grade fractionated coconut oil;
about ¼ cup organic beeswax solid;
about ¼ tbsp organic virgin olive oil;
about ⅛ tsp organic food grade menthol;
about 1/16 tsp sage essential oil;
about 1/16 tsp rosemary essential oil;
about 1/16 tsp peppermint essential oil;
about ⅛ tsp Indian gooseberry extract;
about ⅛ tsp Vitamin E oil;
about 1/16 tsp organic food grade sulfur;
about ⅛ tsp apricot oil;
about ⅛ tsp African organic castor oil;
about ⅛ tsp grape-seed oil; and
about ⅛ tsp liquid Vitamin B complex.

9. The haircare composition of claim 8, further comprising about ⅛ tsp black seed oil.

10. The haircare composition of claim 10, further comprising at least one member selected from the group consisting of magnesium liquid vitamins, sarsaparilla, organic basil, and mixtures thereof.
